(19) United States
(12) Patent Application Publication
(10) Pub. No.: US 2023/0312479 A1
(43) Pub. Date: Oct. 5, 2023

(54) NOVEL AMINO ACID DERIVATIVES

(71) Applicant: PeptiDream Inc., Kawasaki-shi (JP)

(72) Inventors: Haruaki Kurasaki, Kawasaki-shi (JP); Masatoshi Matsumoto, Kawasaki-shi (JP); Ayumu Matsuda, Kawasaki-shi (JP); Yutaka Kobayashi, Kawasaki-shi (JP); Masahiko Kinebuchi, Kawasaki-shi (JP); Tomoko Ashizawa, Kawasaki-shi (JP); Katsuma Matsui, Kawasaki-shi (JP); Motoki Murai, Kawaski-shi (JP); Masami Yamada, Kawaski-shi (JP)

(21) Appl. No.: 18/127,215

(22) Filed: Mar. 28, 2023

Related U.S. Application Data
(60) Provisional application No. 63/325,031, filed on Mar. 29, 2022.

(51) Int. Cl. C07D 233/64; C07C 271/22; C07C 317/28; C07D 239/26; C07D 263/32; C07D 295/088; C07D 307/79; C07D 207/09; C07D 213/73; C07C 311/06

(52) U.S. Cl. CPC C07D 233/64; C07C 271/22; C07C 317/28; C07D 239/26; C07D 263/32; C07D 295/088; C07D 307/79; C07D 207/09; C07D 213/73; C07C 311/06; C07B 2200/07; C07C 2603/18

(57) ABSTRACT

A novel amino acid derivative is provided, wherein the amino acid derivative is expected to improve binding affinity of polypeptides comprising the derivative therein.

### FIELD OF THE DISCLOSURE

This disclosure relates to novel amino acid derivatives.

### BACKGROUND OF THE DISCLOSURE

Recently peptides are paid attention to as pharmaceutical substances. Such peptides interact with the pharmaceutical target molecule to act and exhibit any pharmacological effect. One of the important features needed for these peptides is binding affinity. The strength of affinity varies on target and the purpose of such pharmaceuticals.

The binding affinity of a polypeptide is primarily dependent on the physicochemical properties of its amino acids. To improve affinity, there is a demand for novel amino acid having various type of functional groups on their side chains to fit the structure of target molecules.

### SUMMARY OF THE DISCLOSURE

Generally, however, demand for polypeptides with improved binding affinity remains high.

In one aspect, a novel amino acid derivative which is expected to improve the binding affinity of polypeptides is provided.

In another aspect, a novel amino acid derivative wherein one or more functional groups within the amino acid are protected by protecting groups is provided.

In a further aspect, an enantiomer of the novel amino acid derivative is provided.

### DETAILED DESCRIPTION OF THE DISCLOSURE

### [Abbreviation]

AcOH acetic acid (CAS: 64-19-7)
aq. aqueous
Boc tert-butoxycarbonyl
Boc₂O di-tert-butyl dicarbonate (CAS: 24424-99-5)
br broad
CPME cyclopentyl methyl ether (CAS: 5614-37-9)
CSA 10-Camphorsulfonic acid (CAS: 5872-08-2)
d doublet
dd double doublet
DCE 1,2-dichloroethane (CAS: 107-06-2)
DIAD Diisopropyl azodicarboxylate (CAS: 2446-83-5)
DIC N,N-Diisopropylcarbodiimide (CAS: 693-13-0)
DIPEA, DIEA N,N-diisopropylethylamine (CAS: 7087-68-5)
DMAP 4-dimethylaminopyridine (CAS: 1122-58-3)
DMF N,N-dimethylformamide (CAS: 68-12-2)
EDCl·HCl N-(3-Dimethylaminopropyl)-N′-ethylcarbodiimide hydrochloride (CAS: 25952-53-8)
EtOAc ethyl acetate (CAS: 141-78-6)
Et ethyl
Et₃N triethylamine (CAS: 121-44-8)
Fmoc 9-fluorenylmethoxycarbonyl
Fmoc-OSu 2,5-dioxopyrrolidin-1-yl 9H-fluoren-9-ylmethyl carbonate (CAS: 82911-69-1)
h hour
IPE diisopropyl ether (CAS: 108-20-3)
i-PrOH 2-propanol, isopropanol (CAS: 67-63-0)
M molar
m multiplet
MeOH methanol (CAS: 67-56-1)
min minute
MSA N-trimethylsilyl-N-methylacetamide (CAS: 7449-74-3)
n-BuLi norma/-butyllithium (CAS: 109-72-8)
Pbf (2,2,4,6,7-pentamethyl-2,3-dihydro-1-benzofuran-5-yl)sulfonyl
Pd₂(dba)₃ Tris(dibenzylideneacetone)dipalladium(0) (CAS: 51364-51-3)
Pd₂(dba)₃·CHCl₃ Tris(dibenzylideneacetone)dipalladium(0)-chloroform adduct (CAS: 52522-40-4)
Pd(PPh₃)₂Cl₂ Bis(triphenylphosphine)palladium(II) dichloride (CAS: 13965-03-2)
Pd/C palladium on carbon
rt room temperature
s singlet
satd. saturated
SPhos dicyclohexyl(2′,6′-dimethoxy[1,1′-biphenyl]-2-yl)phosphane (CAS: 657408-07-6)
SPhos Pd G3 (2-Dicyclohexylphosphino-2′,6′-dimethoxybiphenyl) [2-(2′-amino-1,1′-biphenyl)]palladium(II) methanesulfonate (CAS: 1445085-82-4)
t triplet
TEA Triethylamine (CAS: 121-44-8)
tert tertiary
TFA trifluoroacetic acid (CAS: 76-05-1)
THF tetrahydrofuran (CAS: 109-99-9)
TMSCl Chlorotrimethylsilane (CAS: 75-77-4)
Trt triphenylmethyl
AcOH acetic acid (CAS: 64-19-7)
aq. aqueous

### [A Novel Amino Acid Derivative]

The novel amino acid derivative of this disclosure is at least one selected from the group consisting of:

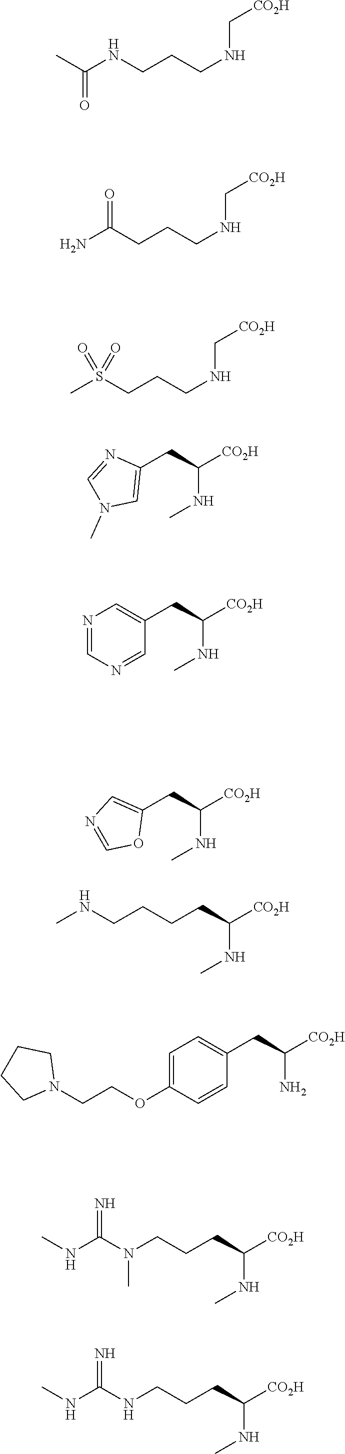

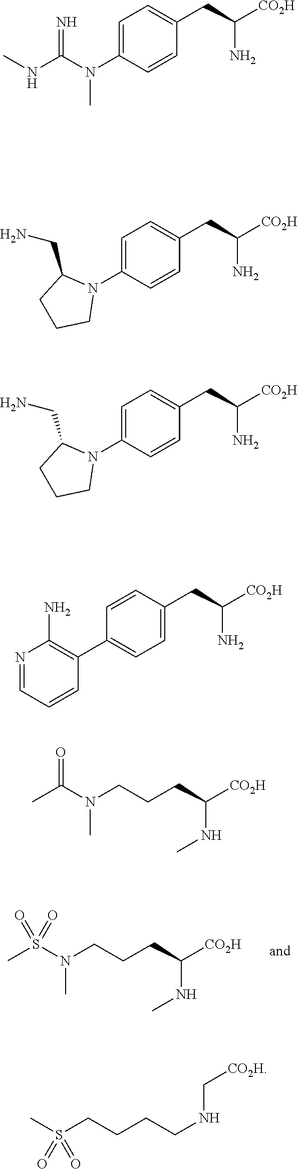

In other words, the novel amino acid derivative of this disclosure is at least one selected from the group consisting of 2-[(3-acetamidopropyl)amino]acetic acid, 2-[(3-carbamoylpropyl)amino]acetic acid, 2-[(3-methanesulfonylpropyl)amino]acetic acid, 3-(1-methyl-1h-imidazol-4-yl)-2-(methylamino)propanoic acid, 2-(methylamino)-3-(pyrimidin-5-yl)propanoic acid, 2-(methylamino)-3-(1,3-oxazol-5-yl)propanoic acid, 2,6-bis (methylamino)hexanoic acid, 2-amino-3-{4-[2-(pyrrolidin-1-yl)ethoxy]phenyl}propanoic acid, 5-(n,n′-dimethylcarbamimidamido)-2-(methylamino)pentanoic acid, 2-(methylamino)-5-(n′-methylcarbamimidamido)pentanoic acid, 2-amino-3-[4-(n,n′-dimethylcarbamimidamido)phenyl]propanoic acid, 2-amino-3-{4-[(2s)-2-(aminomethyl)pyrrolidin-1-yl]phenyl}propanoic acid, 2-amino-3-{4-[(2r)-2-(aminomethyl)pyrrolidin-1-yl]phenyl}propanoic acid, 2-amino-3 4-[4-(2-aminopyridin-3-yl)phenyl]propanoic acid, 2-(methylamino)-5-(n-methylacetamido)pentanoic acid, 2-(methylamino)-5-(n-methylmethanesulfonamido)pentanoic acid, and 2-[(4-methanesulfonylbutypamino]acetic acid.

In one aspect, the novel amino acid derivative of this disclosure may contain any functional group such as carboxyl group and amino groups and any functional group in their side chains protected by conventional or any known protecting groups (e.g. Fmoc, Trt, Pdf, or Boc).

### <Exemplary Description of Synthesizing the Amino Acid Derivatives>

### Example-1: 2-[(3-acetamidopropyl)({[(9H-fluoren-9-yl)methoxy]carbonyl})amino]acetic acid

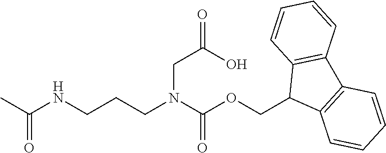 [SMILES: CC(=O)NCCCN(CC(=O)O)C(=O)OCC1c2ccccc2-c2ccccc21]

To a solution of N-(3-aminopropyl)acetamide hydrochloride (CAS: 53186-44-0, 2.00 g, 13.15 mmol) in MeOH (60 mL) at rt was added TEA (5.50 g, 54.35 mmol). The mixture was stirred for 1 h at room temperature (rt) and the solid wasfiltered out. The filtrate was concentrated. To the mixture at 0° C. were added MeOH and a solution of 2-oxoacetic acid hydrate (1.21 g, 13.15 mmol) in MeOH (10 mL) dropwise. The mixture was stirred at rt for 1 h. To the mixture was added Pd/C (2.00 g). The mixture was stirred overnight at rt under H₂ atmosphere. The mixture was filtered out and the filtrate was concentrated. To a mixture of the obtained material in dioxane (500 mL) and water (16 mL) at 0° C. were added Na₂CO₃ (2.70 g, 25.26 mmol) and a suspension of Fmoc-OSu (4.30 g, 12.63 mmol) indioxane (8 mL) dropwise. The resulting mixture was stirred at rt for 2 h. The pH of the mixture was adjusted to 6 with 20% aq. citric acid. The mixture was extracted with EtOAc and washed with brine. The organic extract was dried over Na₂SO₄, filtered, and then concentrated. The resulting residue was purified by silica gel flash column chromatography (gradient, CH₂Cl₂/MeOH=100/1 to 1/1) to afford the title compound (1.47 g, 3.70 mmol). ¹H NMR (300 MHz, MeOH-d₄) δ: 7.85-7.75 (m, 2H), 7.67-7.55 (m, 2H), 7.45-7.25 (m, 4H), 4.59-4.49 (m, 1H), 4.44-4.34 (m, 1H), 4.32-4.13 (m, 1H), 3.99-3.85 (m, 2H), 3.43-3.23 (m, 1H), 3.22-3.03 (m, 2H), 3.00-2.85 (m, 1H), 1.97-1.83 (m, 3H), 1.79-1.62 (m, 1H), 1.54-1.34 (m, 1H). MS (ESI+): m/z 397.1 [M+H].

### Example-2: 2-({[(9H-fluoren-9-yl)methoxy]carbonyl}({3-[(triphenylmethyl)carbamoyl]propyl}amino)acetic acid

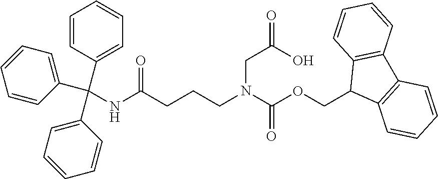 [SMILES: O=C(O)CN(CCCC(=O)NC(c1ccccc1)(c1ccccc1)c1ccccc1)C(=O)OCC1c2ccccc2-c2ccccc21]

To a solution of benzyl N-(3-carbamoylpropyl)carbamate (CAS: 35821-20-6, 4.46 g, 18.88 mmol) in AcOH (63 mL) at rt were added acetic anhydride (3.90 g, 37.75 mmol), triphenylmethanol (9.80 g, 37.75 mmol) and sulfuric acid (0.95 mL). The mixture was stirred at 50° C. for 3 h. To the mixture was added ice-cold water (200 mL). The precipitate was collected. To a solution of the product in MeOH (80 mL) was added Pd/C (4.60 g). The mixture was stirred at rt for 2 h under H₂ atmosphere. The mixture was filtered and the filtrate was concentrated. To a solution of the product (2.10 g, 6.10 mmol) in MeOH (80 mL) and CH₂Cl₂ (12 mL) at 0° C. was added a solution of 2-oxoacetic acid hydrate (0.73 g, 7.93 mmol) in MeOH (8 mL) dropwise. The mixture was stirred at rt for 1 h. To the mixture was added Pd/C (2.10 g). The mixture was stirred at rt overnight under H₂ atmosphere. The reaction mixture was filtered and the filtrate was concentrated. To a mixture of the product (2.45 g, 5.69 mmol) at 0° C. were added Na₂CO₃ (1.20 g,11.38 mmol) and Fmoc-OSu (1.90 g, 5.69 mmol) in dioxane (8 mL) dropwise.

The mixture was stirred at rt for 2 h. The pH of the solution was adjusted to 6 with 20% aq. citric acid. The mixture was extracted with EtOAc and washed with brine. The organic extract was dried over Na₂SO₄, filtered, and then concentrated.

The resulting residue was purified by silica gel flash column chromatography (gradient, petroleum ether/EtOAc=100/1 to 1/1) to afford the title compound (1.01 g, 1.62 mmol). ¹H NMR (300 MHz, MeOH-d₄) δ: 7.83-7.73 (m, 2H), 7.64-7.55 (m, 2H), 7.44-7.14 (m, 19H), 4.54-4.47 (m, 1H), 4.42-4.34 (m, 1H), 4.29-4.15 (m, 1H), 3.90-3.80 (m, 2H), 3.36-3.23 (m, 1H), 3.06-2.95 (m, 1H), 2.36-2.25 (m, 1H), 2.10-1.99 (m, 1H), 1.83-1.67 (m, 1H), 1.57-1.41 (m, 1H). MS (ESI+): m/z 625.2 [M+H].

### Example-3: 2-({[(9H-fluoren-9-yl)methoxy]carbonyl}(3-methanesulfonylpropyl)amino)acetic acid

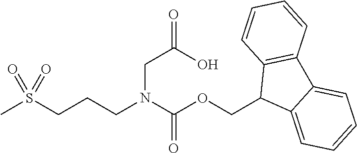 [SMILES: CS(=O)(=O)CCCN(CC(=O)O)C(=O)OCC1c2ccccc2-c2ccccc21]

To a solution of 3-(methylsulfonyl)propan-1-amine hydrochloride (CAS: 157825-88-2, 15.00 g, 86.36 mmol) in CH₃CN (350 mL) at 0° C. were added DIEA (33.50 g, 259.07 mmol) and tert-butyl 2-bromoacetate (13.47 g, 69.09 mmol) dropwise.

The mixture was allowed to warm to rt and stirred at rt overnight. To the mixture was added Boc₂O (37.69 g, 172.72 mmol) at 0° C. The mixture was allowed to warm to rt and stirred at rt overnight. The mixture was concentrated. The resulting residue was purified by silica gel flash column chromatography (gradient, petroleum ether/EtOAc=5/1 to 1/1). Appropriate fractions were concentrated. To the obtained material (9.50 g, 27.03 mmol) in CH₂Cl₂ at rt was added TFA (40 mL). The mixture was stirred at rt for 2 h. The mixture was concentrated and azeotroped with toluene. To a mixture of the product in dioxane (90 mL) and H₂O (45 mL) at 0° C. were added NaHCO₃ (9.10 g, 108.00 mmol) and Fmoc-OSu (8.65 g, 25.65 mmol). The mixture was allowed to warm to rt and stirred for 16 h. The pH of the solution was adjusted to 6 with 20% aq. citric acid. The mixture was extracted with EtOAc and washed with brine. The organic extract was dried over Na₂SO₄, filtered, and then concentrated. The resulting residue was purified by silica gel flashcolumn chromatography (gradient, petroleum ether/EtOAc=5/1 to 1/1) to afford the title compound (5.14 g, 12.3 mmol). ¹H NMR (300 MHz, DMSO-d₆) δ: 12.73 (br s, 1H), 8.07-7.83 (m, 2H), 7.77-7.55 (m, 2H), 7.53-7.22 (m, 4H), 4.50-4.38 (m, 1H), 4.37-4.15 (m, 2H), 4.00 (s, 1H), 3.88 (s, 1H), 3.49-3.26 (m, 1H), 3.24-3.05 (m, 2H), 3.02-2.82 (m, 4H), 1.98-1.84 (m, 1H), 1.82-1.65 (m, 1H). MS (ESI+): m/z 418.1 [M+H].

### Example-4: (2S)-2-({[(9H-fluoren-9-yl)methoxy]carbonyl}(methyl)amino)-3-(1-methyl-1H-imidazol-4-yl)propanoic acid

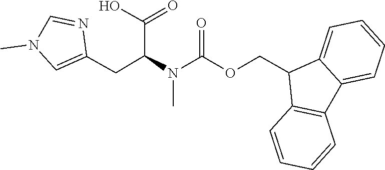 [SMILES: CN(C(=O)OCC1c2ccccc2-c2ccccc21)[C@@H](Cc1cn(C)cn1)C(=O)O]

To a solution of (2S)-2-[[(9H-fluoren-9-ylmethoxy) carbonyl]amino]-3-(1-methylimidazol-4-yl) propanoic acid (CAS: 202920-22-7, 18.00 g, 45.99 mmol) in AcOH (180 mL) at rt were added paraformaldehyde (13.80 g, 459.54 mmol) and CSA (2.14 g, 9.21 mmol). The mixture was allowed to warm to 85° C. and stirred for 2 h. The mixture was concentrated. The resulting residue was purified by reverse-phase flash column chromatography (Column, C18 silica gel; gradient, CHCN/H₂O with 0.1% NH₄HCO₃=7/13 to 13/7). To a mixture of the product (6.30 g, 15.6 mmol) in DCE (35 mL) and TFA (35 mL) at rt was added Et₃SiH (18.30 g, 157.38 mmol). The mixture was stirred at 50° C. for 12 h. The mixture was concentrated. The resulting residue was purified by reverse-phase flash colum n chromatography (Column, C18 silica gel; gradient, CH₃CN/H₂O with 0.1% NH₄HCO₃=3/7 to 3/2) to afford the title compound (5.13 g, 12.67 mmol).

¹H NMR (400 MHz, DMSO-d₆) δ: 7.94-7.83 (m, 2H), 7 -4.14 (m, 3H), 3.62-3.49 (m, 3H), 3.09-2.99 (m, 1H), 2.98-2.84 (m, 1H), 2.84-2.75 (m, 3H). MS (ESI+): m/z 406.0 [M+H].

### Example-5: (2S)-2-({[(9H-fluoren-9-yl)methoxy]carbonyl}(methyl)amino)-3-(pyrimidin-5-yl)propanoic acid

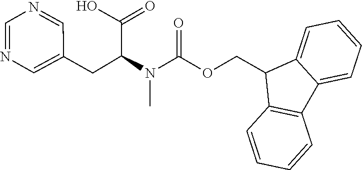 [SMILES: CN(C(=O)OCC1c2ccccc2-c2ccccc21)[C@@H](Cc1cncnc1)C(=O)O]

To a suspension of Zn (9.81 g, 150.05 mmol) in DMF (240 mL) at rt was added 12 (0.95 g, 3.75 mmol). The mixture was stirred for 5 min. To the mixture at rt was added (2R)-2-[(tert-butoxycarbonyl)amino]-3-iodopropanoate (CAS: 93267-04-0, 19.75 g, 60.01 mmol). The mixture was stirred at rt for 0.5 h. To the mixture were added 5-bromopyrimidine_{(7.95 g}, 50.00 mmol), Pd₂(dba)₃ (1.55 g, 1.50 mmol), SPhos (1.03 g, 2.50 mmol) and DMF (60 mL). The mixture was stirred at 50° C. for 4 h.

The reaction mixture was filtered, and then the filtrate was diluted with EtOAc. The resulting mixture was washed with brine, and then dried over Na₂SO₄, filtered, and the n concentrated. The resulting residue was purified by silica gel flash column chromatography (gradient, petroleum ether/EtOAc=1/0 to 3/2). Appropriate fractions were concentrated. To the obtained material (8.40 g, 29.86 mmol) were added THF (90 mL) and H₂O (30 mL). The mixture was stirred at rt for 5 min.

To the solution at rt was added lithium hydroxide (6.27 g, 149.43 mmol). The mixture was stirred at rt for 3 h. The pH value of the mixture was adjusted to 6 with 20% aq. citric acid. The mixture was extracted with EtOAc and washed with brine. The organic extract was dried over Na₂SO₄, filtered, and then concentrated.

The resulting residue was purified by silica gel flash column chromatography (gradient, CH₂Cl_{2/MeOH=}1/0 to 24/1).

Appropriate fractions were collected and concentrated. To a mixture of the obtained material in THF (500 mL) at 0° C. was added NaH (60%, 7.78 g, 194.52 mmol). The mixture was stirred at 0° C. for 0.5 h. To the mixture was added iodomethane (33.14 g, 233.46 mmol). The resulting reaction mixture was stirred at rt for 48 h. The reaction was quenched at 0° C. with water. The pH value of the mixture was adjusted to 6 with 20% aq. citric acid. The mixture was extracted with EtOAc and washed with brine. The organic extract was dried over Na₂SO₄, filtered, and then concentrated. The resulting residue was purified by silica gel flash column chromatography (gradient, CH₂Cl₂/MeOH=1/0 to 97/3). Appropriate fractions were collected and concentrated. To a mixture of the obtained material (7.50 g, 26.66 mmol) in CH₂Cl₂ (40 mL) at rt was added TFA (40 mL). The reaction mixture was stirred at rt for 1 h then concentrated. The residue (26.66 mmol) was dissolved in dioxane (90 mL) and H₂O (30 mL). To the mixture at rt were added NaHCO₃ (4.48 g, 53.33 mmol) and Fmoc-OSu (13.49 g, 39.99 mmol). The mixture was stirred at rt for 3 h. The pH of the solution was adjusted to 6 with 20% aq. citric acid. The mixture was extracted with EtOAc and washed with brine. The organic extract was dried over Na₂SO₄, filtered, and then concentrated. The resulting residue was purified by reverse-phase flash column chromatography (Column, C18 silica gel; gradient, CH₃CN/H₂O=1/9 to 3/7) to afford the title compound (3.53 g, 8.75 mmol). ¹H NMR (400 MHz, DMSO-d₆) δ 13.1 (br s, 1H), 9.07-9.02 (m, 1H), 8.70-8.54 (m, 2H), 7.93-7.84 (m, 2H), 7.61-7.26 (m, 6H), 4.914.66 (m, 1H), 4.27-4.13 (m, 3H), 3.30-3.24 (dd, J=14.8, 5.2 Hz, 0.7H), 3.17-3.07 (m, 1H), 2.90-2.80 (m, 0.3H), 2.77-2.67 (m, 3H). MS (ESI+): m/z 404.1 [M+H].

### Example-6: (2S)-2-({[(9H-fluoren-9-yl)methoxy]carbonyl}(methyl)amino)-3-(1,3-oxazol-5-yl)propanoic acid

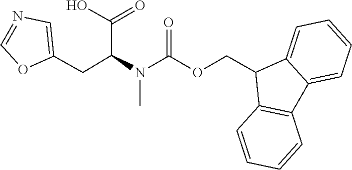 [SMILES: CN(C(=O)OCC1c2ccccc2-c2ccccc21)[C@@H](Cc1cnco1)C(=O)O]

To a solution of ethyl 1,3-oxazole-5-carboxylate (CAS: 118994-89-1, 50.00 g, 354.29 mmol) in EtOH (500 mL) at 0° C. was added NaBH₄ (27.00 g, 713.66 mmol). The mixture was stirred at rt for 12 h. The pH value of the mixture was adjusted to 3 with 3M aq. HCl at 0° C. The mixture was concentrated and the residue was purified by silica gel flash column chromatography (isocratic, petroleumether/EtOAc=1/1). Appropriate fractions were concentrated. The resulting residue (22.40 g, 226.06 mmol) was dissolved in CH₂Cl₂ (200 mL). To the solution at 0° C. was addeddropwise SOCl₂ (40.00 g, 336.22 mmol). The mixture was stirred at rt for 12 h. The pH value of the mixture was adjusted to 8 with NaHCO₃. The mixture was extracted with EtOAc. The organic extract was dried over Na₂SO₄, filtered, and then concentrated to afford the crude material. To a solution of (3R)-3-isopropyl-2,5-dimethoxy-3,6-dihydropyrazine (CAS: 109838-85-9, 29.80 g, 161.75 mmol) in THF (300 mL) at −78° C. was added dropwise n-BuLi (2.5 M in hexane, 78.00 mL). The mixture was stirred at −78° C. for 1 h. To the solution at −78° C. was added dropwise a solution of the crude material (22.80 g, 193.99 mmol) in THF (100 mL). The resulting mixture was stirred at rt for 12 h. The reaction was quenched with satd. aq. NH₄Cl. The mixture was extracted with EtOAc and the organic extract was washed with brine. The extract was dried over Na₂SO₄, filtered, and then concentrated. The residue was purified by silica gel flashcolumn chromatography (isocratic, petroleum ether/EtOAc=3/1). Appropriate fractions were concentrated. The obtained material (23.00 g, 86.69 mmol) was dissolved in CH₃CN (100 mL). To the solution at 0° C. was added dropwise a solution of HCl (20 mL) in THF (80 mL). The resulting mixture was stirred at rt for 12 h and then concentrated. To the resulting residue was dissolved in THF (50 mL) and H₂O (50 mL) at rt was added lithium hydroxide (4.22 g, 176.22 mmol). The reaction mixture was stirred at rt for 4 h and then concentrated. The residue (86.69 mmol) was dissolved in dioxane (300 mL) and H₂O (100 mL). To the solution at 0° C. were added NaHCO₃ (14.85 g, 176.77 mmol) and Fmoc-Cl (22.86 g, 88.38 mmol). The mixture was stirred at rt for 2 h. The pH value of the mixture was adjusted to 5 with citric acid. The mixture was extracted with EtO Ac.

The organic extract was washed with brine, dried over Na₂SO₄, filtered, and then concentrated. The residue was purified by reverse-phase flash column chromatography (Column, C18 silica gel; gradient, CH₃CN/H₂O with 0.1% formic acid=2/3 to 7/3). Appropriate fractions were concentrated. The obtained material (13.30 g, 35.15 mmol) was suspended at rt in toluene (500 mL). To the suspensionwas added paraformaldehyde (10.60 g, 352.98 mmol) and CSA (1.63 g, 7.02 mmol). The resulting mixture was stirred at 100° C. for 3 h.

The mixture was concentrated and then purified by reverse-phase flash column chromatography (Column, C18 silica gel; gradient, CH₃CN/H₂O with 0.1% formic acid=2/3 to 7/3). Appropriate fractions were concentrated. To the obtained material (6.10 g, 15.63 mmol) in TFA (30 mL) at 0° C. was added dropwise Et₃SiH (30.00 mL, 185.76 mmol). The resulting reaction mixture was stirred at rt for 36 h and concentrated. The residue was dissolved in EtOAc. The pH value of the solution was adjusted to 7 with satd. aq. NaHCO₃. The mixture was extracted with EtOAc. The extract was dried over Na₂SO₄, filtered, and then concentrated.

The residue was purified by reverse-phase flash column chromatography (Column, C18 silica gel; gradient, CH₃CN/H₂O with 0.1% formic acid=2/3 to 7/3) to afford the title compound (3.69 g, 9.40 mmol). ¹H NMR (400 MHz, DMSO-d₆) δ 13.1 (br s, 1H), 8.22 (s, 1H), 7.92-7.85 (m, 2H), 7.617.53 (m, 2H), 7.46-7.39 (m, 2H), 7.37-7.28 (m, 2H), 6.93-6.79 (m, 1H), 4.82-4.69 (m, 1H), 4.38-4.17 (m, 3H), 3.29-3.03 (m, 2H), 2.80-2.72 (m, 3H). MS (ESI+): m/z 393.1 [M+H].

### Example-7: (2S)-6-{[(tert-butoxy)carbonyl](methyl)amino}-2-({[(9H-fluoren-9-yl)methoxy]carbonyl}(methyl)amino)hexanoic acid

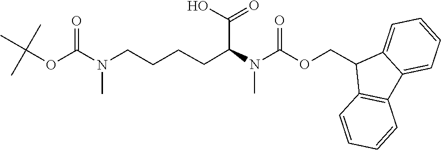 [SMILES: CN(CCCC[C@@H](C(=O)O)N(C)C(=O)OCC1c2ccccc2-c2ccccc21)C(=O)OC(C)(C)C]

To a solution of (2S)-6-(Benzyloxycarbonylamino)-2-(tert-butoxycarbonylamino) hexanoic acid (CAS: 2389-60-8, 1.14 g, 3.00 mmol) in THF (30 mL) at 0° C. was added NaH (60%, 0.42 g, 10.50 mmol). The mixture was stirred at 0° C. for 0.5 h. To the mixture at 0° C. was added dropwise iodomethane (0.87 g, 6.15 mmol).

The resulting mixture was stirred at rt overnight. The mixture was diluted with EtOAc and quenched at 0° C. with 2M aq. H₂SO₄. The mixture was extracted with EtOAc and the separated organic extract was washed with brine, dried over Na₂SO₄, filtered, and then concentrated. The residue was purified by silica gel flashcolumn chromatography (gradient, CH₂Cl_{2/}MeOH=1/0 to 4/1). Appropriate fractions were concentrated. To the resulting residue (0.88 g, 2.14 mmol) at rt wereadded toluene (4.3 mL), Pd/C (10%, 0.23 g, 0.214 mmol) and ammonium formate (0.54 g, 8.57 mmol). The resulting mixture was stirred at 100° C. for 3 h. The mixture was filtered and the filtrate was concentrated. The residue was washed with IPE and then collected by filtration. The residue (0.48 g, 1.76 mmol) was dissolved in acetone (8.8 mL) and H₂O (8.8 mL). To the mixture at rt were added NaH CO₃ (0.37 g, 3.52 mmol) and Fmoc-OSu (0.71 g, 2.11 mmol). The mixture was stirred at rt for 12 h. The mixture was concentrated and the pH value of the residue was adjusted to 2 with 2M aq. HCl. The mixture was extracted with EtOAc and washed with brine. The organic extract was dried over Na₂SO₄, filtered, and then concentrated. The resulting residue was purified by silica gel flash column chromatography (gradient, CH₂Cl₂/MeOH=1/0 to 17/3) to afford the title compound (0.67 g, 1.35 mmol). ¹HNMR (500 MHz, DMSO-d₆) δ 12.8 (br s, 1H), 7.94-7.85 (m, 2H), 7.70-7.58 (m, 2H), 7.45-7.38 (m, 2H), 7.36-7.27 (m, 2H), 4.55-4.18 (m, 4H), 3.18-3.05 (m, 2H), 2.77-2.66 (m, 6H), 1.89-0.94 (m, 15H). MS (ESI+): m/z 497.4 [M+H].

### Example-8: (2S)-2-({[(9H-fluoren-9-yl)methoxy]carbonyl}amino)-3-{4-[2-(pyrrolidin-1-yl)ethoxy]phenyl}propanoic acid hydrochloride

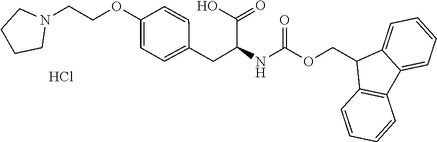 [SMILES: O=C(N[C@@H](Cc1ccc(OCCN2CCCC2)cc1)C(=O)O)OCC1c2ccccc2-c2ccccc21 ; Cl]

To a solution of triphenylphosphine (13.32 g, 50.79 mmol) in THF (100 mL) at 0° C. was added DIAD (10.27 g, 50.79 mmol). The mixture was stirred at 0° C. for 10 min. To the mixture at rt were added methyl (25)-2-[(tert-butoxycarbonyl)amino]-3-(4-hydroxyphenyl) propanoate (CAS: 4326-36-7, 10.00 g, 33.86 mmol) and 2-(pyrrolidin-1-yl)ethan-1-ol (5.85 g, 50.79 mmol). The resulting reaction mixture was stirred at rt for 14 h. The reaction was quenched with H₂O. The mixture was extracted with EtOAc and the organic extract was dried over Na₂SO₄, filtered, and then concentrated. The residue was purified by silica gel flash column chromatography (isocratic, CH₂Cl₂/MeOH=9/1). Appropriate fractions were concentrated. To the obtained material (13.30 g, 33.86 mmol) at rt were added CH₂Cl₂ (133 mL) and TFA (66 mL). The mixture was stirred at rt for 0.5 h and then concentrated. The residue (9.90 g, 33.86 mmol) was dissolved in dioxane (120 mL)and H₂O (40 mL). To the mixture were added NaHCO₃ (14.22 g, 169.30 mmol) and Fmoc-OSu (11.42 g, 33.86 mmol). The mixture was stirred at rt for 1 h. The mixture was diluted with H₂O and extracted with EtOAc. The organic extract was dried over Na₂SO₄, filtered, and then concentrated. The resulting residue was purified by silica gel flash column chromatography (isocratic, CH₂Cl₂/MeOH=9/1). Appropriate fractions were concentrated. The obtained material (6.00 g, 11.66 mmol) was dissolved in DCE. To the mixture at rt was added trimethyltin hydroxide (10.54 g, 58.30 mmol). The resulting reaction mixture was stirred at 40° C. for 14 h. The mixture was diluted with H₂O and the pH value of the mixture was adjusted to 3 with 1M aq. HCl.

The mixture was extracted with EtOAc and the organic extract was dried over Na₂SO₄, filtered, and then concentrated. The crude residue was purified by reverse-phase flash column chromatography (Column, C18 silica gel; gradient, CH₃CN/H₂O=1/19 to 1/0) to afford the title compound (1.61 g, 2.99 mmol). ¹H NMR (300 MHz, DMSO-d₆) δ 12.8 (br s, 1H), 10.8 (br s, 1H), 7.91 (d, J=7.5 Hz, 2H), 7.74 (d, J=8.6 Hz, 1H), 7.70-7.63 (m, 2H), 7.43 (dd, J=7.4 Hz, 2H), 7.38-7.20 (m, 4H), 6.99-6.89 (m, 2H), 4.38-4.10 (m, 6H), 3.64-3.49 (m, 4H), 3.15-2.77 (m, 4H), 2.08-1.79 (m, 4H). MS (ESI+): m/z 499.1 [M−H]

### Example-9: (2S)-5-[(E)-N,N′-dimethyl-N″-[(2,2,4,6,7-pentamethyl-2,3-dihydro-1-benzofuran-5-yl)sulfonyl]carbamimidamido]-2-({[(9H-fluoren-9-yl)methoxyl]carbonyl}(methyl)amino)pentanoic acid

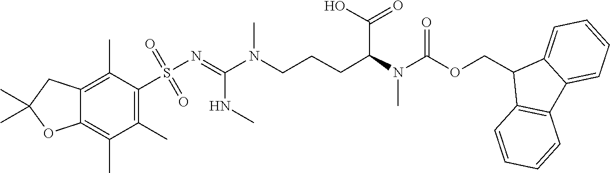 [SMILES: CN/C(=N\S(=O)(=O)c1c(C)c(C)c2c(c1C)CC(C)(C)O2)N(C)CCC[C@@H](C(=O)O)N(C)C(=O)OCC1c2ccccc2-c2ccccc21]

To a solution of 2,2,4,6,7-pentamethyl-2,3-dihydrobenzofuran-5-sulfonamide (CAS: 378230-81-0, 3 g, 11.14 mmol) in DMF (30 mL) at 0° C. was added NaH (0.47 g, 11.69 mmol) and stirred at rt for 15 min. Then isothiocyanatomethane (0.80 g, 10.91 mmol) was added and stirred at 50° C. for 30 min. The mixture was cooled at rt then diluted with water. The mixture was acidified with 2M aq. HCl. The mixture was extracted with a mixture of EtOAc and hexane. The organic layer was washed with water and brine, dried over Na₂SO₄, filtered, and concentrated in vacuo to afford 3.05 g of 2,2,4,6,7-pentamethyl-N-(methylcarbamothioyl)-2,3-dihydrobenzofuran-5-sulfonamide (Intermediate-1). To the solution of (S)-2-((((9H-fluoren-9-yl)methoxy)carbonyl)(methyl)amino)-5-((tert-butoxycarbonyl)(methyl)amino)pentanoic acid (CAS: 2389078-95-7, 2.00 g, 4.14 mmol) in CH₂Cl₂ (13.8 mLmL) at 0° C. was added 4M HCl in CPME (4.1 mL, 16.58 mmol). The resulting mixture was stirred at rt for 2 h and then concentrated in vacuo to afford 1.77 g of (S)-2-4((9H-fluoren-9-yl)methoxy)carbonyl)(methyl)amino)-5-(methylamino)pentanoic acid hydrochloride (Intermediate-2).

To a suspension of Intermediate-2 (500 mg, 1.19 mmol) in THF (7.5 mL) was added MSA (0.57 mL, 3.58 mmol) and stirred at rt for 30 min until the suspension turned into a clear colorless solution. To the solution at rt were added Intermediate-1 (0.41 g, 1.19 mmol), which was prepared in the procedure described above, and EDCl·HCl (0.24 g, 1.25 mmol). The mixture was stirred at rt for 1 h. The reaction was quenched with 10% aq. citric acid and the mixture was extracted with EtOAc.

The organic layer was washed with water and brine, dried over Na₂SO₄, filtered and concentrated in vacuo to give a white amorphous solid. This white solid was dissolved in hot EtOAc and heptane was added to the solution. The resulting white precipitate was collected and washed with EtOAc/heptane (15/85) to afford the title compound (0.76 mg, 1.10 mmol). ¹H NMR (500 MHz, DMSO-d₆) δ 12.80 (s, 1H), 8.00-7.78 (m, 2H), 7.61 (td, J=10.6, 9.0, 4.2 Hz, 2H), 7.52-7.21 (m, 4H), 7.08 (q, J=4.6 Hz, 1H), 4.58-4.03 (m, 4H), 3.15 (q, J=8.6, 7.8 Hz, 2H), 2.91 (s, 2H), 2.86-2.71 (m, 6H), 2.61 (s, 3H), 2.45 (s, 3H), 2.37 (d, J=2.1 Hz, 3H), 1.98 (s, 3H), 1.37 (d, J=2.3 Hz, 10H). MS (ESI+): m/z 691.4 [M+H].

### Example-10: (2S)-2-({[(9H-fluoren-9-yl)methoxy]carbonyl}(methyl)amino)-5-[(E)-N′-methyl-N″-[(2,2,4,6,7-pentamethyl-2,3-dihydro-1-benzofuran-5-yl)sulfonyl]carbamimid amido]pentanoic acid

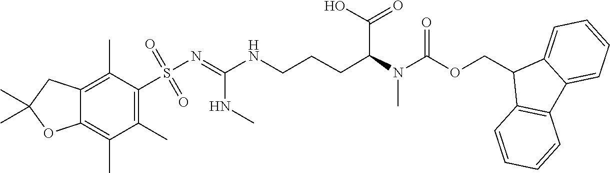 [SMILES: CN/C(=N\S(=O)(=O)c1c(C)c(C)c2c(c1C)CC(C)(C)O2)NCCC[C@@H](C(=O)O)N(C)C(=O)OCC1c2ccccc2-c2ccccc21]

A round-bottomed flask charged with (S)-2-((((9H-fluoren-9-yl)methoxy)carbonyl)(methyl)amino)-5-((tert-butoxycarbonyl)amino)pentanoic acid (CAS: 1793105-28-8, 4.00 g, 8.54 mmol) was added CH₂Cl₂ (42.7 mL) and 4 M HCl in CPME (42.7 mL) at rt. The mixture was stirred at rt for 1 h then concentrated in vacuo to afford 3.62 g of (S)-2-((((9H-fluoren-9-yl)methoxy)carbonyl)(methyl)amino)-5-aminopentanoic acid hydrochloride (Intermediate-3).

To a suspension of Intermediate-3 (0.50 g, 1.24 mmol) in THF (7.5 mL) was added MSA (0.59 mL, 3.70 mmol) and stirred at rt for 30 min until the suspension turned into a clear colorless solution. To the solution at rt were added Intermediate-1 (0.42 g, 1.24 mmol), which was prepared in the procedure described above (Example 9), and EDCl·HCl (0.25 g, 1.30 mmol). The mixture was stirred at rt for 1 h. The reaction was quenched with 10% aq. citric acid, then the mixture was extracted with EtOAc. The organic layer was washed with water and brine, dried over Na₂SO₄, filtered and concentrated in vacuo. The crude material was purified by silica gel flash column chromatography (gradient, CH₂Cl₂/MeOH=1/0 to 0/1) to afford the title compound (0.44 g, 1.24 mmol) to afford the title compound. ¹H NMR (500 MHz, DMSO-d₆) δ 12.85 (s, 1H), 7.91-7.85 (m, 2H), 7.64-7.58 (m, 2H), 7.43-7.27 (m, 4H), 7.10 (s, 1H), 4.41-4.19 (m, 4H), 3.18-3.09 (m, 2H), 2.97-210 (m, 15H), 2.02-1.98 (m, 3H), 1.77-1.15 (m, 10H). MS (ESI+): m/z 677.4 [M+H].

### Example-11: (2S)-3-{4-[(E)-N,N′-dimethyl-N″-[(2,2,4,6,7-pentamethyl-2,3-dihydro-1-benzofuran-5-yl)sulfonyl]carbamimidamido]phenyl}-2-({[(9H-fluoren-9-yl)methoxy]carbonyl}amino)propanoic acid

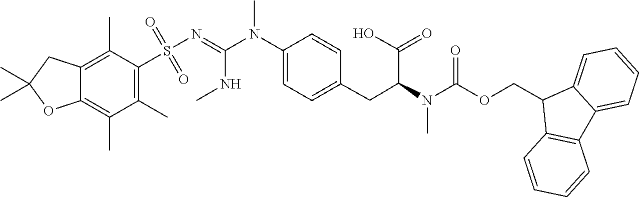 [SMILES: CN/C(=N\S(=O)(=O)c1c(C)c(C)c2c(c1C)CC(C)(C)O2)N(C)c1ccc(C[C@@H](C(=O)O)N(C)C(=O)OCC2c3ccccc3-c3ccccc32)cc1]

A three-necked round-bottomed flask fitted with a N₂ balloon was charged with Zn (1.47 g, 22.51 mmol). The flask was heated with a heat gun under high vacuum and backfilled with N₂ gas. To the flask at rt were added DMF (7.50 mL), 1,2-dibromoethane (0.065 mL, 0.75 mmol) and TMSCl (0.10 mL, 0.75 mmol), and the mixture was allowed to warm to 60° C. gradually. After stirring at 60° C. for 30 min, to the mixture was added a solution of methyl (R)-2-((((9H-fluoren-9-yl) methoxy)carbonyl)amino)-3-iodopropanoate (CAS: 156017-42-4, 6.77 g, 15.01 mmol) in DMF (7.5 mL) at rt. After stirring at rt for 1.5 h, the consumption of the starting material was monitored by LC-MS. The resulting mixture was settled at rt over 30 min.

An another three-necked round bottomed flask was charged with tert-butyl (4-iodophenyl)(methyl)carbamate (CAS: 306768-12-7, 2.50 g, 7.50 mmol) and SPhosPd G3 (0.59 g, 0.75 mmol). The flask was charged with DMF (22.5 mL). To the mixture at 50-60° C. was transferred the solution of the organozinc reagent, which was prepared in the procedure described above by using a syringe. After stirring at 60° C. for 3 h, the reaction was diluted with EtOAc, and quenched with 2 M aq. HCl at 0° C. After stirring at 0° C. for 5 min, the mixture was filtered through a celite pad.

The filtrate was diluted with EtOAc, then washed with brine. The organic extract was dried over Na₂SO₄, filtered, and concentrated under reduced pressure.

The crude material was purified by silica gel flash column chromatography (gradient, hexane/EtOAc=1/0 to 0/1). To a solution of the obtained material (1.00 g, 1.89 mmol) in DCE (9.4 mL) at rt was added trimethyltin hydroxide (1.02 g, 5.65 mmol). The resulting mixture was stirred at 40° C. for 22 h then concentrated.

The crude material was purified by silica gel flash column chromatography (with 10 wt % KF/silica gel; gradient, CH₂Cl₂/MeOH=97/3 to 5/1). A round-bottomed flask fitted with a N₂ balloon was charged with the obtained material (1.00 g, 1.94 mmol). To the flask at rt were added CH₂Cl₂ (9.68 mL) and 4 M HCl in CPME (9.68 mL). After stirring at rt for 1 h, the mixture was concentrated. The crude material was washed with IPE, dried at 50° C. under reduced pressure. To a suspension of the obtained material (0.60 g, 1.33 mmol) in THF (7.5 mL) at rt was added MSA (0.64 mL, 3.97 mmol) and the mixture was stirred for 30 min until the suspension turned into a clear colorless solution. To the solution at rt was added Intermediate-1 (0.45 gg, 1.33 mmol), which was prepared in the procedure described above (Example 9), and EDCl·HCl (0.27 g, 1.39 mmol). The mixture was stirred at rt for 1 h then the reaction was quenched with 10% aq. citric acid. The mixture was extracted with EtOAc. The organic layer was washed with water and brine, dried over Na₂SO₄, filtered and concentrated in vacuo. The residue was dissolved in hot EtOAc and heptane was added to the solution. The resulting white precipitate was collected and washed with EtOAc/heptane (15/85) to afford the title compound (0.51 g, 0.71 mmol). ¹H NMR (500 MHz, DMSO-d₆) δ 2.81 (s, 1H), 7.88 (d, J=7.5 Hz, 2H), 7.75 (d, J=8.6 Hz, 1H), 7.67-7.52 (m, 2H), 7.41 (t, J=7.4 Hz, 2H), 7.33-7.21 (m, 4H), 7.14-7.03 (m, 3H), 4.23-4.13 (m, 4H), 3.36-2.82 (m, 10H), 2.43 (s, 3H), 2.28-2.21 (m, 3H), 2.02 (s, 3H), 1.41 (s, 6H). MS (ESI+): m/z 725.4 [M+H].

### Example-12: (2S)-3-{4-[(2S)-2-({[(tert-butoxy)carbonyl]amino}methyl)pyrrolidin-1-yl]phenyl}-2-({[(9H-fluoren-9-yl)methoxy]carbonyl}amino)propanoic acid

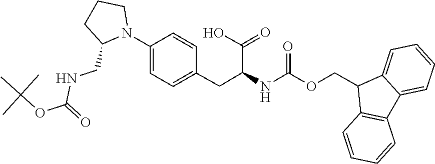 [SMILES: CC(C)(C)OC(=O)NC[C@@H]1CCCN1c1ccc(C[C@H](NC(=O)OCC2c3ccccc3-c3ccccc32)C(=O)O)cc1]

To a round bottomed flask at rt were added 1,4-diiodobenzene (32.94 g, 99.86 mmol), to rt-butyl N-[(2S)-pyrrolidin-2-ylmethyl]carbamate (CAS: 141774-70-1, 10.00 g, 49.93 mmol), 1,1′-bi-2-naphthol (2.86 g, 9.99 mmol), CuI (1.90 g, 9.99 mmol), K₃PO₄ (21.21 g, 99.86 mmol), DMF (200 mL) under N₂ atmosphere. The resulting solution was stirred overnight at 80° C. The reaction was then quenched by the addition of water. The resulting mixture was extracted with EtOAc. The organic layer was washed with brine, dried over Na₂SO₄ and concentrated. The residue was purified by silica gel flash column chromatography (isocratic, petroleum ether/EtOAc=1/1) to yield 6.00 g of tert-butyl N-[[(2S)-1-(4-iodophenyl)pyrrolidin-2-yl]methyl]carbamate as a yellow solid. A three-necked round-bottomed flask fitted with a N₂ balloon was charged with Zn (4.04 g, 62.15 mmol). The flask was heated with a heat gun under high vacuum and backfilled with N₂ gas. The flask was charged with DMF (50 mL), 1,2-dibromoethane (233.5 mg, 1.24 mmol) and TMSCl (270.1 mg, 2.49 mmol) at rt. After stirring at 60° C. for 30 min, to the mixture at rt was added methyl (2R)-2-[[(9H-fluoren-9-ylmethoxy)carbonyl]amino]-3-iodopropanoate (CAS: 156017-42-4, 6.73 g, 14.92 mmol). The mixture was stirred at rt for 1 h then settled over 30 min at rt. An another three-necked round bottomed flask was charged with Pd₂(dba)₃ (0.64 g, 0.62 mmol), SPhos (0.51 g, 1.24 mmol), and DMF(50 mL) at rt. The mixture was stirred at rt for 1 h to give a dark yellow solution of the Pd complex. An another three-necked round-bottomed flask fitted with a N₂ balloon was charged with tert-butyl N-[[(2S)-1-(4-iodophenyl)pyrrolidin-2-yl]methyl]carbamate (5.00 g, 12.43 mmol). The flask was charged with DMF (50 mL), the solution of the Pd complex and the solution of the organozinc reagent at rt. After stirring at 30° C. overnight, the mixture was diluted with EtOAc, and quenched with water at rt. After stirring for 5 min, the mixture was filtered. The filtrate was diluted with EtOAc and washed with brine, dried over Na₂SO₄, filtered, and concentrated under reduced pressure. The crude material was purified by flash column chromatography (isocratic, petroleum ether/EtOAc=3/1). Appropriate fractions were concentrated. The obtained material (5.00 g, 8.34 mmol) was dissolved in i-PrOH (150 mL) and H₂O (150 mL). The resulting solution was cooled at 0° C. To the mixture at rt were added CaCl₂ (9.25 g, 83.37 mmol) and LiOH·H₂O (2.80 g, 66.70 mmol). The resulting mixture was stirred at 30° C. overnight. The reaction was then quenched by the addition of water. The resulting mixture was extracted with EtOAc. The organic layer was washed with brine, dried over Na₂SO₄ and concentrated. The residue was purified by silica gel flash column chromatography (isocratic, petroleum ether/EtOAc=1/1) and reverse-phase flash column chromatography (Column, C18 silica gel; gradient, CH₃CN/H₂O=1/19 to 1/0) to afford the title compound (1.07 g, 1.83 mmol).

¹H NMR (300 MHz, DMSO-d₆) δ 7.91-7.88 (m, 2H), 7.70-7.55 (m, 2H), 7.60-7.20 (m, 4H), 7.20-6.90 (m, 4H), 6.80-6.50 (m, 2H), 4.304.10 (m, 3H), 4.00-3.90 (m, 1H), 3.60-3.50 (m, 2H), 3.40-3.25 (m, 1H), 3.20-3.10 (m, 1H), 3.00-2.85 (m, 1H), 2.81-2.70 (5 m, 1H), 2.69-2.51 (m, 1H), 2.00-1.60 (m, 4H), 1.40-1.30 (m, 9H). MS (ESI+): m/z 586. 30 [M+H].

The following compound was synthesized as outlined for the preparation of Example-12 employing appropriate starting materials (tert-butyl N-[(2R)-pyrrolidin-2-ylmethyl]carbamate, CAS: 719999-54-9).

### Example-13: (2S)-3-{4-[(2R)-2-({[(tert-butoxy)carbonyl]amino}methyl)pyrrolidin-1-yl]phenyl}-2-({[(9H-fluoren-9-yl)methoxy]carbonyl}amino)propanoic acid

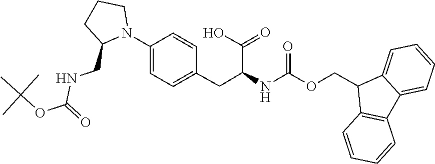 [SMILES: CC(C)(C)OC(=O)NC[C@H]1CCCN1c1ccc(C[C@H](NC(=O)OCC2c3ccccc3-c3ccccc32)C(=O)O)cc1]

¹H NMR (300 MHz, DMSO-d₆) δ 7.90 (d, J=7.1 Hz, 2H), 7.72-7.63 (m, 2H), 7.45-7.35 (m, 2H), 7.35-7.20 (m, 3H), 7.20-6.95 (m, 3H), 6.63 (d, J=7.4 Hz, 2H), 4.20-4.01 (m, 4H), 3.59 (s, 1H), 3.35-3.15 (s, 2H), 3.05-2.85 (m, 2H), 2.75-2.65 (m, 2H), 1.93-1.78 (m, 4H), 1.43 (s, 9H). MS (ESI+): m/z 586.30 [M+H].

### Example-14: (2S)-3-[4-(2-aminopyridin-3-yl)phenyl]-2-({[(9H-fluoren-9-yl)methoxy]carbonyl}amino)propanoic acid

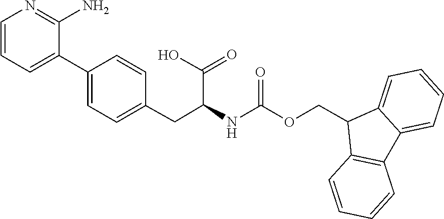 [SMILES: Nc1ncccc1-c1ccc(C[C@H](NC(=O)OCC2c3ccccc3-c3ccccc32)C(=O)O)cc1]

To a round bottomed flask at rt were added 3-iodopyridin-2-amine (CAS: 104830-06-0, 10.00 g, 45.45 mmol), 4-bromophenylboric acid (10.95 g, 54.54 mmol), Na₂CO₃ (9.59 g, 90.45 mmol), Pd(PPh₃)₂Cl₂ (4.79 g, 6.82 mmol), DME (300 mL) and H₂O (100 mL). The resulting mixture was stirred at 50° C. for 1 h.

The reaction was then quenched by the addition of water (100 mL) and extracted with EtOAc The organic extract was dried over Na₂SO₄ and concentrated. The resulting residue was purified by silica gel flash column chromatography (isocratic, petroleum ether/EtOAc=4/1) to afford 3-(4-bromophenyl)pyridin-2-amine as a yellow solid. To a three-necked round bottomed flask at rt were added Zn (3.91 g, 59.81 mmol), DMF (50 mL), 1,2-dibromoethane (0.23 gg, 1.20 mmol) and TMSCl (0.26 gg, 2.41 mmol) under N₂ atmosphere. After stirring at 60° C. for 30 min, to the mixture at rt was added methyl (2R)-2-[[(9H-fluoren-9-ylmethoxy)carbonyl]amino]-3-iodopropanoate (CAS: 156017-42-4, 6.47 g, 14.33 mmol). The resulting mixture was stirred at rt for 1 h. To a three-necked round bottom ed flask at rt were added Pd₂(dba)₃·CHCl₃ (0.62 g, 0.60 mmol), SPhos (0.49 g, 1.20 mmol), and DMF (50 mL). The mixture was stirred at rt for 1 h to give a dark yellow solution of the Pd complex. Into a three-necked round bottomed flask were added at rt 3-(4-bromophenyl) pyridin-2-amine (3.00 g, 12.04 mmol), DMF (50 mL), the solution of the Pd complex and the solution of the organozinc reagent. The resulting mixture was stirred at 30° C. overnight. The mixture was diluted with EtOAc, and quenched with water at rt.

After stirring for 5 min, the mixture was filtered. The filtrate was extracted with EtOAc then the organic extract was washed with brine, dried over Na₂SO₄ and concentrated. The resulting residue was purified by silica gel flash column chromatography (isocratic, petroleum ether/EtOAc=5/1). Appropriate fractions were concentrated. To a mixture of the obtained material (3.00 g, 6.08 mmol), i-PrOH (90 mL) and H₂O (30 mL) at 0° C. were added CaCl₂ (6.75 g, 60.78 mmol) and LiOH (0.73 g, 30.39 mmol). The resulting solution was stirred at 30° C. for 12 h.

The pH value of the solution was adjusted to 5 with 1M aq. HCl. The mixture was extracted with EtOAc and the organic extract was washed with brine, dried over Na₂SO₄, filtered and concentrated. The resulting residue was purified by silica gel flash column chromatography (isocratic, petroleum ether/EtOAc=3/1) to afford the title compound (1.03 g, 2.15 mmol).

¹H NMR (300 MHz, DMSO-d₆) δ 12.95 (br s, 1H), 7.94-7.65 (m, 6H), 7.42-7.25 (m, 9H), 6.64 (dd, J=7.0, 4.9 Hz, 1H), 5.56 (s, 2H), 4.23-4.17 (m, 4H), 3.15-2.82 (m, 2H). MS (ESI+): m/z 480.20 [M+H].

### Example-15: (2S)-2-({[(9H-fluoren-9-yl)methoxy]carbonyl}(methyl)amino)-5-(N-methylacetamido)pentanoic acid

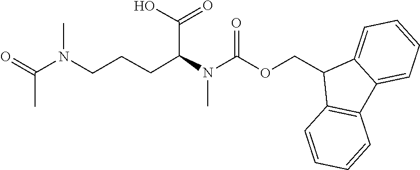 [SMILES: CC(=O)N(C)CCC[C@@H](C(=O)O)N(C)C(=O)OCC1c2ccccc2-c2ccccc21]

4M HCl in CPME (5.23 mL, 20.93 mmol) was added to (S)-2-((((9H-fluoren-9-yl)methoxy)carbonyl)(methyl)amino)-5-((tert-butoxycarbonyl)(methyl)amino)pentanoic acid (CAS: 2389078-95-7, 5.00 g, 10.36 mmol), and the reaction mixture was stirred at rt for 2 h. The reaction mixture was concentrated to afford 4.63 g of (S)-2-((((9H-fluoren-9-yl)methoxy)carbonyl)(methyl)amino)-5-(methylamino)pentanoic acid hydrochloride (Intermediate-4). Acetic anhydride (1.32 mL, 13.99 mmol) was added to a solution of 2.93 g of Intermediate-4 in THF (35 mL) and Water (35 mL) at 0° C., and the reaction mixture was stirred at rt for 2 h. The reaction mixture was quenched with 1M aq. HCl to adjust pH to 1, and the mixture was extracted with EtOAc. The organic extract was dried over Na₂SO₄, filtered and concentrated. The residue was purified by silica gel flash column chromatography (isocratic, CH₂Cl_{2/MeOH=}91/9) and reverse-phase flash column chromatography (Column, C18 silica gel; gradient, CH₃CN with 0.5% TFA/H₂O=1/2 to 1/1) to afford the title compound (1.47 g, 3.46 mmol).

¹H NMR (500 MHz, DMSO-d₆) δ 12.82 (br s, 1H), 7.91-7.88 (m, 2H), 7.66-7.62 (m, 2H), 7.44-7.27 (m, 4H), 4.54-4.25 (m, 4H), 3.29-3.11 (m, 2H), 2.90-2.71 (m, 6H), 1.97-1.24 (m, 7H). MS (ESI+): m/z 425.4 [M+H].

### Example-16: (2S)-2-({[(9H-fluoren-9-yl)methoxy]carbonyl}(methyl)amino)-5-(N-methylmethanesulfonamido)pentanoic acid

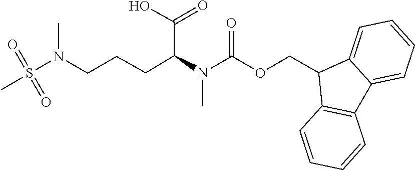 [SMILES: CN(C(=O)OCC1c2ccccc2-c2ccccc21)[C@@H](CCCN(C)S(C)(=O)=O)C(=O)O]

Trimethylsilyl (E)-N-(trimethylsilyl)acetimidate (7.94 mL, 32.5 mmol) was added to a solution of 1.70 g of Intermediate-4, which was prepared in the procedure described above (Example 15), in THF (20 mL) at 0° C., and the reaction mixture was stirred at 0° C. for 2 h. To the mixture at 0° C. was added methanesulfonyl chloride (1.26 mL, 16.23 mmol), then the mixture was stirred at rt for 2 h. The reaction mixture was quenched with 1M aq. HCl to adjust pH to 1, and the mixture was extracted with EtOAc. The organic extract was dried over Na₂SO₄, filtered and concentrated, then the residue was purified by silica gel flash column chromatography (isocratic, CH₂Cl₂/MeOH=10/1) and reverse-phase flash column chromatography (Column, C18 silica gel; gradient, CH₃CN with 0.5% TFA/H₂O=1/2 to 1/1) to afford the title compound (1.09 g, 2.37 mmol).

¹H NMR (500 MHz, DMSO-d₆) δ 12.83 (br s, 1H), 7.91-7.89 (m, 2H), 7.67-7.63 (m, 2H), 7.44-7.32 (m, 4H), 4.53-4.25 (m, 4H), 3.18-2.70 (m, 8H), 1.88-1.24 (m, 7H). MS (ESI+): m/z 461.0 [M+H].

### Example-17: 2-({[(9H-fluoren-9-yl)methoxy]carbonyl}(4-methanesulfonylbutyl)amino) acetic acid

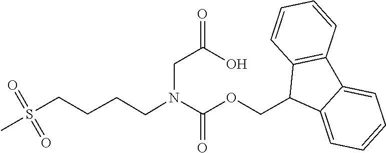 [SMILES: CS(=O)(=O)CCCCN(CC(=O)O)C(=O)OCC1c2ccccc2-c2ccccc21]

To a solution of tert-butyl (4-hydroxybutyl)carbamate (CAS: 75178-87-9, 22.00 g, 116.22 mmol) in CH₂Cl₂ (450 mL) at 0° C. was added Et₃N (17.64 g, 174.33 mmol) followed by slow addition of methanesulfonyl chloride (16.00 g, 139.46 mmol). The resulting mixture was stirred at rt for 1 h. The mixture was diluted with CH₂Cl₂ and washed with brine. The separated organic extract was dried over Na₂SO₄, filtered, and then concentrated. To the residue at 0° C. were added DMF (450 mL) and sodium thiomethoxide (12.20 g, 174.33 mmol). The mixture was stirred at rt for 24 h. The mixture was diluted with H₂O then extracted with EtOAc.

The organic extract was washed with brine, dried over Na₂SO₄, filtered, and then concentrated. The residue was purified by silica gel flash column chromatography (gradient, petroleum ether/EtOAc=9/1 to 1/1). Appropriate fractions were concentrated. The obtained material (20.00 g, 91.18 mmol) was dissolved in MeOH (500 mL) and H₂O (500 mL). To the solution at 0° C. was added Oxone® (50.50 g, 145.92 mmol). The mixture was stirred at rt for 2 h. The resulting solid was filtered off and the filtrate was concentrated. The residue was diluted with EtOAc and washed with brine. The organic extract was dried over Na₂SO₄, filtered,and then concentrated. The residue (20.00 g, 79.59 mmol) was dissolved in 4M HCl in dioxane (250 mL) at 0° C. The resulting mixture was stirred at rt for 2 h. The mixture was concentrated and the residue was azeotroped with toluene. To a mixture of the residue in CH₃CN (32 mL) at 0° C. were added DIPEA (31.00 g, 239.73 mmol) followed by tert-butyl 2-bromoacetate (12.47 g, 63.93 mmol). The reaction mixture was stirred at rt for 14 h and then concentrated. The residue was diluted with H₂O and extracted with CH₂Cl₂. The organic extract was washed with brine, dried over Na₂SO₄, filtered, and then concentrated. The residue was purified by silica gel flash column chromatography (gradient, CH₂Cl₂/MeOH=9/1 to 4/1).

Appropriate fractions were concentrated. To a solution of the obtained material (4.30 g, 16.20 mmol) in dioxane (60 mL) and H₂O (30 m) at 0° C. were added NaHCO₃ (4.10 g, 48.60 mmol) and Fmoc-OSu (5.20 g, 15.40 mmol). The resulting mixture was stirred at rt for 16 h. The pH value of the mixture was adjusted to 6 with citric acid. The mixture was extracted with EtOAc. The organic extract was washed with brine, dried over Na₂SO₄, filtered, and then concentrated. The residue was purified by silica gel flash column chromatography (gradient, petroleum ether/EtOAc=17/3 to 1/1). Appropriate fractions were concentrated. The obtained material (5.50 g, 11.28 mmol) was dissolved in 4M HCl in dioxane (60 mL) at 0 ° C. The mixture was stirred at rt for 2 h and then concentrated. The residue was azeotroped with toluene and purified by silica gel flash column chromatography (gradient, petroleum ether/EtOAc=7/3 to 1/1) to afford the title compound (3.11 g, 7.21 mmol). NMR (300 MHz, DMSO-d₆) δ 12.7 (br s, 1H), 7.90 (d, J=7.5 Hz, 2H), 7.70-7.58 (m, 2H), 7.47-7.27 (m, 4H), 4.48 (d, J=5.4 Hz, 1H), 4.35-4.17 (m, 2H), 3.98 (s, 1H), 3.83 (s, 1H), 3.34-3.24 (m, 1H), 3.18-3.07 (m, 1H), 3.01-2.90 (m, 5H), 1.76-1.17 (m, 4H). MS (ESI+): m/z 432.1 [M+H].

### [Polypeptide]

The polypeptide of this disclosure comprises at least one amino acid described herein above.

What is claimed is:

1. An amino acid derivative selected from the group consisting of:

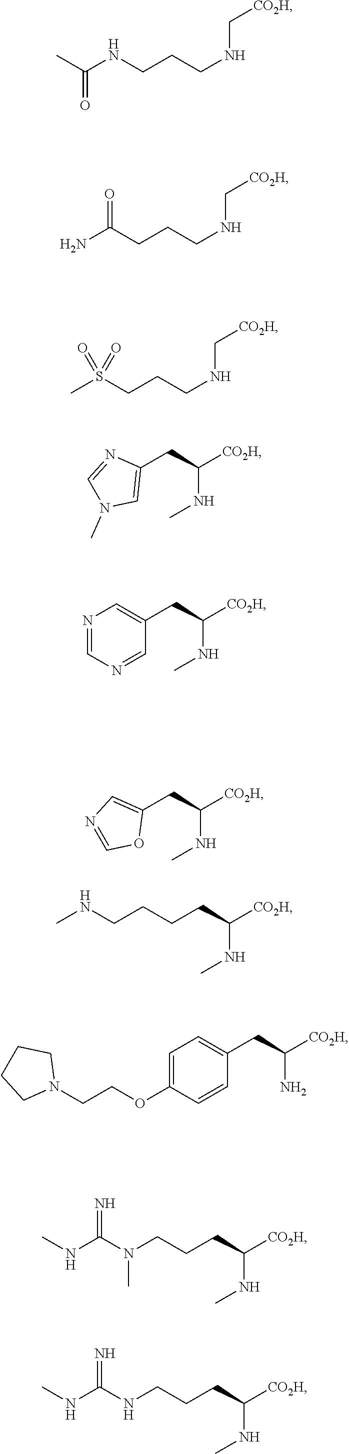

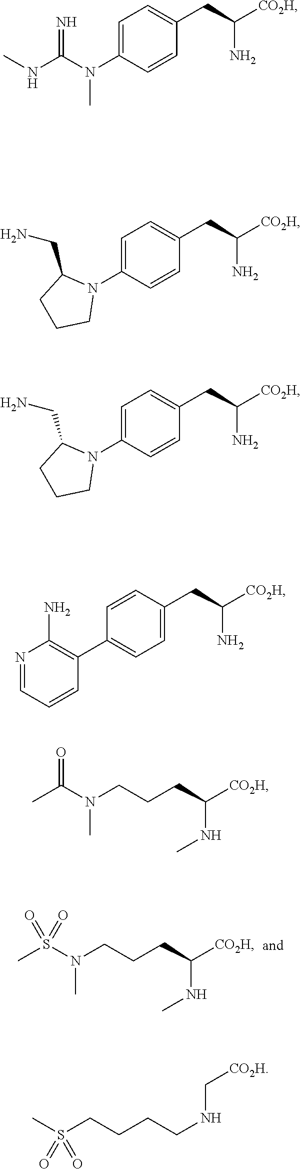

2. An amino acid derivative selected from the group consisting of 2-[(3-acetamidopropyl)amino]acetic acid, 2-[(3-carbamoylpropyl)amino]acetic acid, 2-[(3-methanesulfonylpropyl)amino]acetic acid, 3-(1-methyl-1h-imidazol-4-yl)-2-(methylamino)propanoic acid, 2-(methylamino)-3-(pyrimidin-5-yl)propanoic acid, 2-(methylamino)-3-(1,3-oxazol-5-yl)propanoic acid, 2,6-bis(methylamino)hexanoic acid, 2-amino-3-{4-[2-(pyrrolidin-1-yl)ethoxy]phenyl}propanoic acid, 5-(n,n′-dimethylcarbamimidamido)-2-(methylamino)pentanoic acid, 2-(methylamino)-5-(n′-methylcarbamimidamido)pentanoic acid, 2-amino-3-[4-(n,n′-dimethylcarbamimidamido)phenyl]propanoic acid, 2-amino-3-{4-[(2s)-2-(aminomethyppyrrolidin-1-yl]phenyl}propanoic acid, 2-amino-3-{4-[(2r)-2-(aminomethyppyrrolidin-1-yl]phenyl}propanoic acid, 2-amino-3-[4-(2-aminopyridin-3-yl)phenyl]propanoic acid, 2-(methylamino)-5-(n-methylacetamido)pentanoic acid, 2-(methylamino)-5-(n-methylmethanesulfonamido)pentanoic acid, and 2-[(4-methanesulfonylbutyl)amino]acetic acid.

3. The amino acid derivative according to claim 1 or 2, wherein one or more functional groups within the amino acid are protected by protecting groups.

4. An enantiomer of the amino acid derivative according to any one of claims 1 to 3.
